(19) United States
(12) Patent Application Publication
(10) Pub. No.: US 2022/0005112 A1
(43) Pub. Date: Jan. 6, 2022

(54) VEHICLE AND METHOD OF MANAGING CLEANLINESS OF INTERIOR OF THE SAME

(71) Applicants: HYUNDAI MOTOR COMPANY, Seoul (KR); KIA MOTORS CORPORATION, Seoul (KR)

(72) Inventors: Seung Hyun Woo, Seoul (KR); Anastasia Yarygina, Seoul (KR); Min Jae Park, Seongnam-si (KR); Edvin Eriksen, Berlin (DE)

(21) Appl. No.: 17/110,919

(22) Filed: Dec. 3, 2020

(30) Foreign Application Priority Data
Jul. 1, 2020 (KR) 10-2020-0080978

(51) Int. Cl. G06Q 30/06; G06K 9/00; G06Q 30/02; G06Q 50/30

(52) U.S. Cl. CPC G06Q 30/0645; G06Q 50/30; G06Q 30/0207; G06K 9/00832

(57) ABSTRACT

A method of managing cleanliness of an interior of a vehicle includes: detecting indoor contamination using a contamination detector including at least a camera, outputting information on a cleaning request upon detecting a first contaminant as a result of the detecting the indoor contamination, determining whether the first contaminant has been removed when a predetermined condition is satisfied, and imposing a penalty on a user upon determining that the first contaminant has not been removed as a result of the determining.

### CROSS-REFERENCE TO RELATED APPLICATION(S)

This application claims the benefit of Korean Patent Application No. 10-2020-0080978, filed on Jul. 1, 2020 in the Korean Intellectual Property Office, which is hereby incorporated by reference as if fully set forth herein.

### TECHNICAL FIELD

The present disclosure relates to a vehicle and a method of managing cleanliness of an interior of the same that are capable of detecting whether the interior of the vehicle has been contaminated and managing detection information.

### BACKGROUND

While a vehicle is traveling, an object present in the interior of the vehicle is subjected to inertial force due to changes in the road surface or acceleration/deceleration of the vehicle. For this reason, in many cases, an object that may cause contamination, such as food, spills and soils a seat or a mat of the vehicle. In the case in which indoor contamination occurs in a privately owned vehicle, the vehicle owner may freely determine whether or not to clean the vehicle. However, in recent years, the concept of vehicle sharing, rather than individual ownership, has expanded due to the advantages of cost savings and utilization efficiency, and thus vehicle sharing and related service markets are expected to maintain high growth. Accordingly, the need to manage the cleanliness of the interior of a vehicle is on the rise.

In a vehicle-sharing service, many people may share one vehicle. Most users have little or no sense of ownership over a shared vehicle, unlike a privately owned vehicle, and thus pay little attention to indoor contamination while using the shared vehicle. Therefore, in many cases, a vehicle is returned with the interior thereof soiled, and is handed over to the next user with the interior thereof in an uncleaned state, which causes dissatisfaction with service quality.

The information included in this Background section is only for enhancement of understanding of the general background of the present disclosure and may not be taken as an acknowledgement or any form of suggestion that this information forms the prior art already known to a person skilled in the art.

### SUMMARY

The present disclosure is directed to a vehicle and a method of managing the cleanliness of the interior of the same that substantially obviate one or more problems due to limitations and disadvantages of the related art.

An object of the present disclosure is to provide a vehicle and a method of managing the cleanliness of the interior of the same that are capable of more effectively detecting whether the interior of a vehicle has been contaminated and informing an occupant of the detection result.

In addition, another object of the present disclosure is to provide a vehicle and a method of managing the cleanliness of the interior of the same that are capable of inducing an occupant to clean the vehicle based on information on the detected contamination.

However, the objects to be accomplished by the embodiments are not limited to the above-mentioned objects, and other objects not mentioned herein will be clearly understood by those skilled in the art to which the embodiments pertain from the following description.

In order to accomplish the above and other objects, a method of managing the cleanliness of the interior of a vehicle according to an embodiment of the present disclosure may include detecting indoor contamination using a contamination detector including at least a camera, outputting information on a cleaning request upon determining that a first contaminant is present as a result of the detecting the indoor contamination, determining whether the first contaminant has been removed when a predetermined condition is satisfied, and imposing a penalty on a user when the first contaminant has not been removed as a result of the determining.

In addition, a device for managing the cleanliness of the interior of a vehicle according to an embodiment of the present disclosure may include an output unit, a contamination detector including at least a camera, and a controller configured to determine indoor contamination based on information acquired using the contamination detector, to output information on a cleaning request through the output unit when a first contaminant is present, to determine whether the first contaminant has been removed using the contamination detector when a predetermined condition is satisfied, and to perform control such that a penalty is imposed on a user upon determining that the first contaminant has not been removed.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure. In the drawings:

FIG. 1 shows an example of the structure of a device for managing the cleanliness of an interior of a vehicle to which embodiments of the present disclosure are applicable;

FIG. 2 is a flowchart showing an example of a process of managing the cleanliness of an interior of a vehicle according to an embodiment of the present disclosure;

FIG. 3 is a flowchart showing an example of the manner in which the indoor cleanliness detection process of FIG. 2 is performed;

FIGS. 4A and 4B show an example of the manner in which image-based object detection is performed according to an embodiment of the present disclosure;

FIGS. 5A, 5B, and 5C show an example of the manner in which image-based contamination detection is performed according to an embodiment of the present disclosure;

FIGS. 6A and 6B show an example of the manner in which image-based contamination tracking is performed according to an embodiment of the present disclosure;

FIG. 7 shows an example of the form in which cleanliness-based guidance information is output according to an embodiment of the present disclosure;

FIGS. 8A and 8B show an example of contamination guidance information according to an embodiment of the present disclosure;

FIG. 9 shows an example of cleanliness-based guidance information for each section according to an embodiment of the present disclosure;

FIG. 10 is a flowchart showing an example of the process of managing the cleanliness of the interior of a vehicle according to another embodiment of the present disclosure;

FIGS. 11A and 11B show an example of the form in which cleaning request information is output through a terminal of a user according to another embodiment of the present disclosure;

FIGS. 12A, 12B and 12C show an example of the form in which information is output through a terminal of a user depending on whether cleaning has been performed according to another embodiment of the present disclosure;

FIGS. 13A and 13B show an example of the form in which information according to non-performance of cleaning is output through a terminal of a user according to another embodiment of the present disclosure;

FIG. 14 is a flowchart showing an example of the process in which the next user uses a shared vehicle in the state in which a contaminant left behind by the previous user is present in the interior of the shared vehicle according to another embodiment of the present disclosure;

FIGS. 15A, 15B, 15C and 15D show an example of information output through a terminal of a user in the process of using a shared vehicle in which a contaminant remains according to another embodiment of the present disclosure; and

FIG. 16 shows another example of the form in which information is output through a terminal of a user in the process of using a shared vehicle in which a contaminant remains according to another embodiment of the present disclosure.

### DETAILED DESCRIPTION

Hereinafter, embodiments of the present disclosure will be described in detail with reference to the accompanying drawings so that those skilled in the art may easily carry out the embodiments. The present disclosure may, however, be embodied in many different forms, and should not be construed as being limited to the embodiments set forth herein. In the drawings, parts irrelevant to the description of the present disclosure will be omitted for clarity. Like reference numerals refer to like elements throughout the specification.

Throughout the specification, when a certain part “includes” or “comprises” a certain component, this indicates that other components are not excluded, and may be further included unless otherwise noted. The same reference numerals used throughout the specification refer to the same constituent elements.

An embodiment of the present disclosure proposes technology for detecting and analyzing the cleanliness of the interior of a vehicle in various manners and outputting guidance information based thereon.

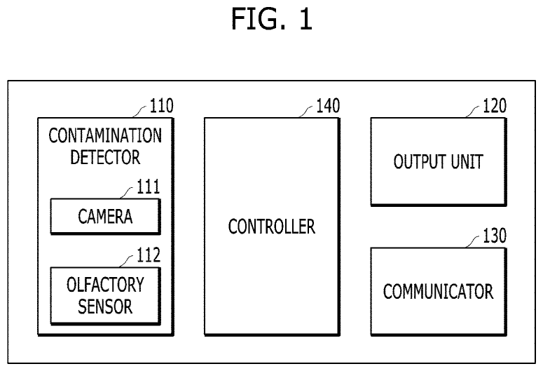

FIG. 1 shows an example of the structure of a device for managing the cleanliness of the interior of a vehicle to which embodiments of the present disclosure are applicable.

Referring to FIG. 1, a device for managing the cleanliness of the interior of a vehicle to which embodiments of the present disclosure are applicable may include a contamination detector 110, which detects whether the interior of the vehicle has been contaminated, an output unit 120, which outputs guidance information, a communicator 130, which performs communication with an internal device and an external device of the vehicle so as to exchange data therewith, and a controller 140, which performs overall control of the aforementioned components.

The contamination detector 110 may include a camera 111 for acquiring an image of the interior of the vehicle in order to detect contamination based on image information and an olfactory sensor 112 for detecting contamination through odor. The camera 111 may include a plurality of cameras depending on the area of the interior that is subjected to contamination detection and on the contamination detection method. For example, the cameras may be disposed at a region near the rear-view mirror in order to photograph the front seats and the surroundings thereof (e.g. the driver's seat, the front passenger seat, the dashboard, etc.) and at the center or the rear end portion of the roof in order to photograph the rear seats and the surroundings thereof (e.g. the rear passenger seats, the lower end region of the rear glass, etc.), without being limited thereto. The position and number of cameras are not limited, so long as it is possible to effectively photograph the indoor region that is being subjected to contamination detection. Similarly, the position and number of olfactory sensors 112 are not limited.

At least one of a general RGB-type camera, an infrared (IR) camera, a stereo camera for capturing a three-dimensional (3D) image, or a camera equipped with a spectroscopic sensor may be used as the camera 111 in order to detect an object and contamination, which will be described later.

The output unit 120 may output guidance information in various forms. For example, the output unit 120 may include a display to output guidance information in the form of visual information, or may include a speaker to output guidance information in the form of acoustic information. The display may include a display of a head unit or a display disposed in a cluster. However, the display is not limited to any specific form, so long as it is capable of visually outputting guidance information.

The communicator 130 may include at least one of a wired communicator (not shown), through which the vehicle cleanliness management device communicates with other entities (e.g. control units) mounted in the vehicle, and a wireless communicator (not shown), through which the vehicle cleanliness management device communicates with entities (e.g. an external server, a smart device carried by an occupant, etc.) other than the corresponding vehicle. The wired communicator may support one or more protocols that are applicable to predetermined vehicle network communication, such as CAN, CAN-FD, LIN, and Ethernet, without being limited thereto. The wireless communicator may support at least one of short-range communication (e.g. Bluetooth, Wi-Fi, Wi-Di, ZigBee, NFC, etc.) for communication with a smart device carried by an occupant, telematics, or cellular-based communication (e.g. 3G, LTE, 5G, etc.).

Upon detecting preparation or commencement of movement of the vehicle through the communicator 130, the controller 140 may obtain information for determination of the indoor cleanliness from the contamination detector 110 to analyze the indoor cleanliness, and may output guidance information corresponding to the analysis result through the output unit 120. In addition, the controller 140 may transmit information corresponding to the analysis result to an external entity, such as a vehicle-sharing service server, a telematics server, or a terminal carried by an occupant, through the communicator 130.

Hereinafter, a process of managing the cleanliness of the interior of a vehicle will be described in detail on the basis of the configuration of the vehicle cleanliness management device described above.

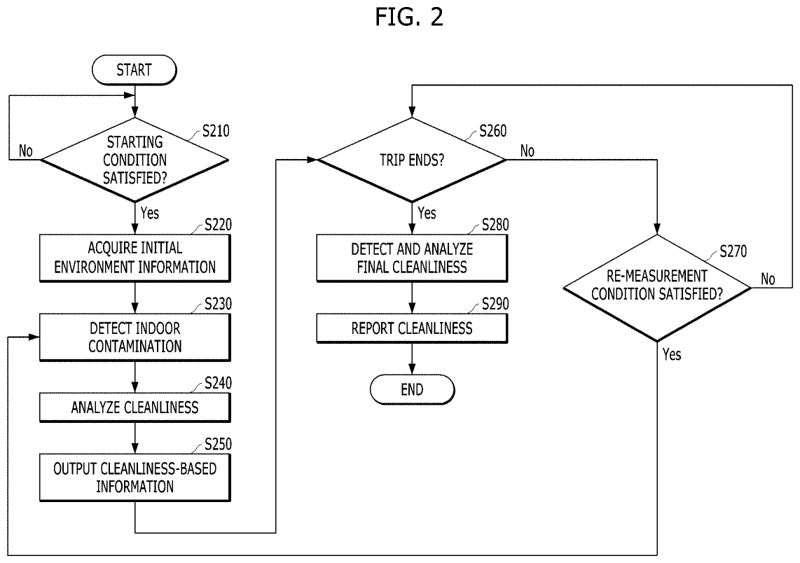

FIG. 2 is a flowchart showing an example of a process of managing the cleanliness of the interior of a vehicle according to an embodiment of the present disclosure.

Referring to FIG. 2, the controller 140 may determine whether a starting condition is satisfied (S210). For example, the starting condition may be satisfied when notification is received from a vehicle-sharing service server that the vehicle is assigned to the next user, when the door of the vehicle is unlocked after the end of the previous trip, or when the vehicle is started, without being limited thereto.

When the starting condition is satisfied (Yes in S210), initial environment information may be acquired (S220). Here, the initial environment information may be information on the state of the interior of the vehicle before the user starts to use the vehicle. For example, the initial environment information may be information on the state of the interior of the vehicle when the vehicle is shipped, information on the state before the current user starts to drive the vehicle after the end of the last use thereof, or information on the state before contamination occurs after the current user gets in the vehicle. Thus, this step may be performed in the manner of acquiring an image for determination of the vehicle interior material, the location of basic in-vehicle items, and the color of the seats and mats through the camera 111 and detecting the odor of the interior before contamination through the olfactory sensor 112. In another embodiment, this step may be performed in the manner of acquiring default initial environment information from a vehicle-sharing service server. In still another embodiment, this step may be omitted.

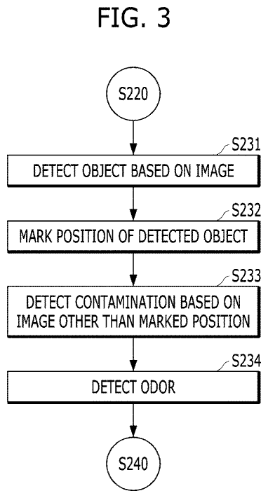

Subsequently, indoor contamination detection may be performed through the contamination detector 111 in order to determine the cleanliness of the interior of the vehicle (S230), and the controller 140 may analyze the cleanliness based on the detection information (S240). A detailed description of the detection process S230 will be made later with reference to FIGS. 3, 4A, 4B, 5A, 5B, 5C, 6A and 6B. The cleanliness may be analyzed in the following manner.

The cleanliness may be analyzed on the basis of the classification and location of contamination.

TABLE 1

| | | Difference | | |
|---|---|---|---|---|
| | | between | | |
| | | Color of | | |
| | Size of | Contaminated | | |
| Type of | Contaminated | Region and | Material | Severity |
| Contaminant | Region | Original Color | Property | of Odor |
| Liquid | 30 cm or | Large | High | High |
| | Greater | Difference | Viscosity | |
| Solid | 5 to 30 cm | Intermediate | Low | Intermediate |
| | | Difference | Viscosity | |
| | Less than 5 cm | Small | No | No Odor |
| | | Difference | Viscosity | |
| | | | (Dry) | |

As can be seen from Table 1, the contamination may be classified on the basis of the type of contaminant, the size of the contaminated region, the difference between the color of the contaminated region and the original color, the material property (viscosity), and the severity of odor. The cleanliness may be given different scores (or weights) depending on the classification of each item. For example, if the size of the contaminated region is 30 cm or greater, the color difference is large, and the severity of odor is high, the cleanliness may be determined to be very low, and if the size of the contaminated region is less than 5 cm, the color difference is small, and there is no odor, the cleanliness may be determined to be very high.

TABLE 2

| Material of Contaminated | Elapsed Time since |
|---|---|
| Region | Contamination Discovery |
| Leather | 60 Minutes or more |
| Fabric | 30 to 60 Minutes |
| Plastic | 10 to 30 Minutes |
| Metal | Within 10 Minutes |

As can be seen from Table 2, the cleanliness may be weighted differently depending on the material of the contaminated region and the contamination time period for each material. For example, in the case of metal, which is relatively resistant to contamination, the cleanliness may not be greatly lowered even when a large amount of time passes after contamination. However, in the case of fabric, which is susceptible to contamination, the cleanliness may be weighted strongly so as to be greatly lowered even when a small amount of time passes after contamination.

In summary, the controller 140 may give a cleanliness score to each item shown in Tables 1 and 2 such that the cleanliness score gradually increases from the uppermost criterion of each item to the lowermost criterion thereof, and may determine the cleanliness by summing the scores given to the respective items. However, this is merely illustrative, and it will be apparent to those skilled in the art that the items/criteria shown in Tables 1 and 2 and the weights given thereto may be variously set.

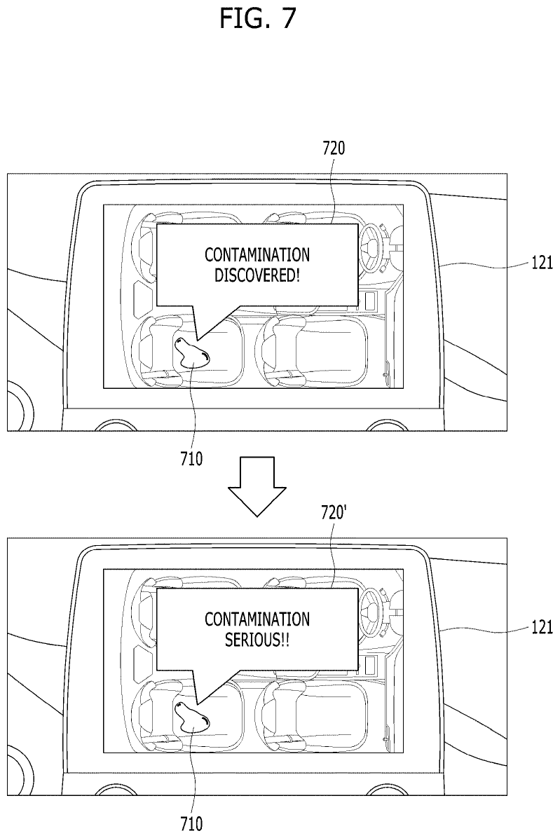

Upon determining that the interior of the vehicle is contaminated based on the cleanliness analysis result, the controller 140 may output guidance information about the indoor contamination through the output unit 120 (S250). A concrete form in which the guidance information is output will be described later with reference to FIGS. 7, 8A, 8B and 9.

The controller 140 may determine whether a re-measurement condition is satisfied (S270) until the end of the trip (No in S260), and may repeatedly detect and analyze the indoor cleanliness when the re-measurement condition is satisfied (Yes in S270). Here, the re-measurement condition may be satisfied when a predetermined amount of time has passed since the last cleanliness analysis, when cleanliness-based guidance indicating that immediate cleaning is required is output, when the contamination level continues to increase at every re-measurement, when at least one of the objects that had been detected previously has disappeared, or when there is a change in occupants through opening of the door, without being limited thereto. For example, among the re-measurement conditions, in the case in which at least one of the objects (including the occupants) that had been detected previously has disappeared, the cleanliness of the region that was hidden or covered by the object that has disappeared may be re-measured.

When the trip ends (Yes in S260), the controller 140 may detect and analyze the final cleanliness through the contamination detector 110 (S280), and may report the analysis result to the outside through the communicator 130 (S290). In some embodiments, when the process of detecting and analyzing the final cleanliness is not separately performed, the most recently analyzed cleanliness information may be reported. The entity to which the cleanliness information is reported may be at least one of a vehicle-sharing service server, a preset cleaning management company server, a telematics center, or a smart device carried by the occupant. When the vehicle-sharing service server receives the cleanliness report, it may assign benefits or a penalty to the last user based on the cleanliness report. When the cleaning management company server receives the cleanliness report, it may determine whether to dispatch cleaning personnel. In addition, the cleanliness report may be transmitted to a smart device carried by the last user in order to warn of the possibility of imposing a penalty on the last user, thereby inducing the last user to return to the vehicle and clean the same. In addition, the cleanliness report transmitted to a smart device carried by the last user may include information indicating that an object that is not a contaminant (i.e. a lost article) was left behind in the vehicle so that the last user may pick up the same immediately.

Hereinafter, the cleanliness detection process will be described in greater detail with reference to FIGS. 3, 4A, 4B, 5A, 5B, 5C, 6A and 6B.

FIG. 3 is a flowchart showing an example of the manner in which the indoor cleanliness detection process of FIG. 2 is performed.

Referring to FIG. 3, in order to detect the indoor cleanliness, object detection may be performed based on an image captured by the camera 111 (S231). The object detection may be a process of detecting an object that was not present in the initial state through object detection logic in an image captured by an RGB camera or a stereo camera. In this case, the controller 140 may directly perform the object detection using preset artificial intelligence (AI) logic, or may transmit an image acquired through the camera 111 to an external server through the communicator 130 and may receive an object detection result from the external server. In addition, in order to detect an object that was not present in the initial state, the controller 140 may compare the currently acquired image with the initial environment information.

Since the present disclosure is not limited to any specific object detection logic, a description of a concrete algorithm of the object detection logic will be omitted.

When at least one object is detected to be present in the interior of the vehicle through the object detection process S231, the controller 140 may mark (i.e. record) the position at which the corresponding object is detected (S232).

In addition, the controller 140 may detect whether the interior of the vehicle has been contaminated based on an image captured by the camera 111 (S233). In this case, the indoor region that is being subjected to contamination detection may be a region other than the region in which an object is detected. The reason for this is to prevent the object itself from being mistaken as contamination. As a contamination detection method, a method of determining whether the inherent color of molecules of indoor components differs from the initial environment information using a spectroscopic sensor may be applied. For example, if both the wavelength corresponding to the inherent color of a leather seat and the wavelength corresponding to another color are detected, it may be determined that the leather seat has been contaminated. In addition, if the reflectivity of a certain part in an image captured by an RGB camera or an IR camera increases, it may be determined that the part has been contaminated by liquid. Apart from the above contamination detection methods, any other contamination detection method may be applied, so long as it is possible to determine contamination through image analysis or optical analysis.

In addition, in order to detect the indoor cleanliness, odor detection may be performed using the olfactory sensor 112 (S234). Although the odor detection process is illustrated in FIG. 3 as being performed in the final stage, it may be performed prior to the image-based detection process, or may be performed simultaneously therewith.

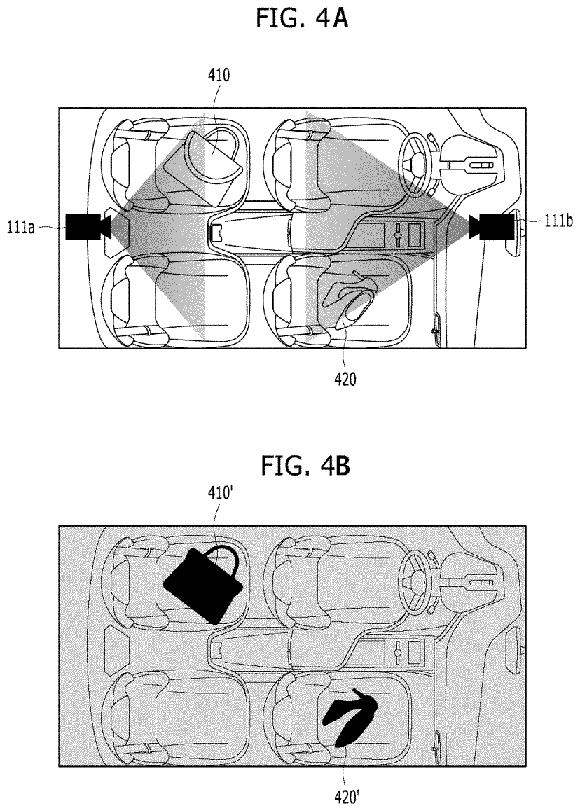

FIGS. 4A and 4B show an example of the manner in which image-based object detection is performed according to an embodiment of the present disclosure.

In FIGS. 4A and 4B and the drawings below, it is assumed that the camera 111 mounted in the interior of the vehicle includes a camera 111a disposed at a region near the rear seats in order to photograph the rear seats and the surroundings thereof and a camera 111b disposed at a region near the front seats in order to photograph the front seats and the surroundings thereof.

Referring to FIG. 4A, an object 410 present on the rear seat behind the driver's seat and an object 420 present on the front passenger seat may be detected through the image-based detection process. The controller 140, as shown in FIG. 4B, may mark positions 410′ and 420′ at which the objects 410 and 420 are respectively detected so that the positions 410′ and 420′ are excluded from the region that is subjected to contamination detection when the image-based contamination detection process is performed.

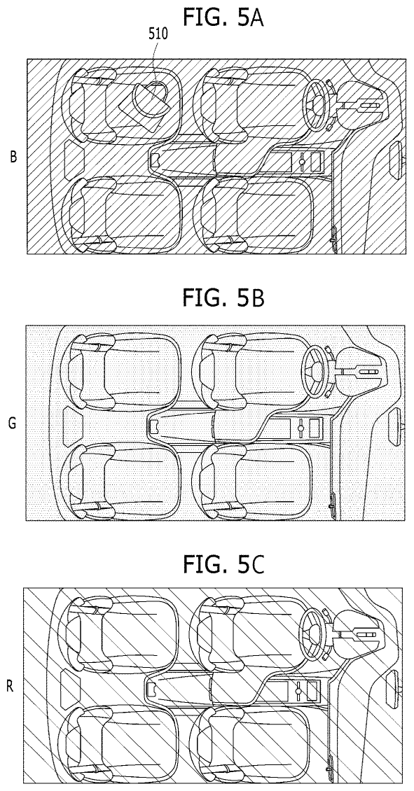

FIGS. 5A, 5B, and 5C show an example of the manner in which image-based contamination detection is performed according to an embodiment of the present disclosure.

FIGS. 5A, 5B, and 5C show an example of an image captured in order to apply a spectroscopy technique to each RGB color region. Here, if there is a region 510 in which a wavelength different from that of the initial environment information is detected, the region 510 may be recognized as a contaminated region. Similar to the detection process of FIGS. 4A and 4B, if an object 410 is detected on the rear seat behind the driver's seat, the region 510 is excluded from the region that is subjected to contamination detection, and thus the difference between the wavelength detected in the region 510 and the initial environment information may be ignored.

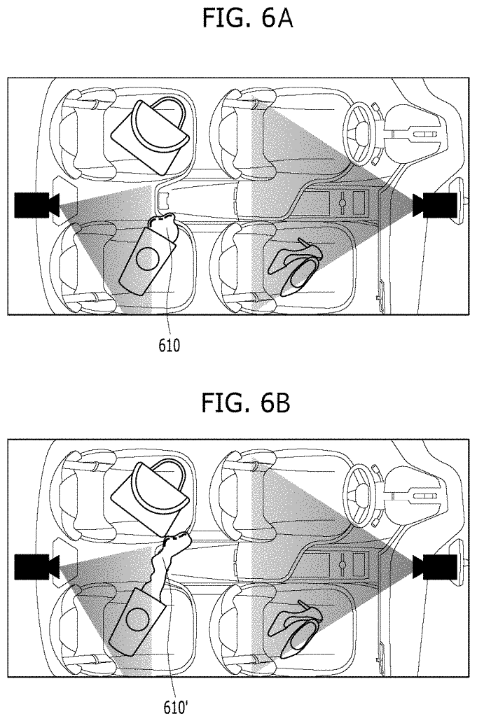

FIGS. 6A and 6B show an example of the manner in which image-based contamination tracking is performed according to an embodiment of the present disclosure.

Referring to FIG. 6A, a contaminated region 610 may be detected in the cleanliness detection process. Thereafter, when the re-measurement condition is satisfied (Yes in S270) and the indoor cleanliness is detected again, if it is detected that the contamination of the region 610 has expanded, as indicated by reference numeral 610′ in FIG. 6B, the controller 140 may determine that the contamination level has increased.

Hereinafter, a form in which the guidance information is output will be described with reference to FIGS. 7, 8A, 8B and 9.

FIG. 7 shows an example of the form in which cleanliness-based guidance information is output according to an embodiment of the present disclosure.

Referring to the upper drawing in FIG. 7, when the controller 140 determines that the indoor cleanliness level determined thereby is equal to or less than a predetermined level, the controller 140 may output guidance information corresponding to the determination through a display 121 of the head unit. Here, the guidance information may include at least one of a display object 710 or text 720, which indicates a contaminated region and the degree of contamination.

When the determined cleanliness is very low or when the size of the contaminated region is increasing as shown in FIGS. 6A and 6B, the controller 140 may change the form of at least some of the guidance information. For example, as shown in the lower drawing in FIG. 7, the content and the color of the text 720′ may be changed, but this is merely illustrative. Various types of visual effects may be provided depending on the cleanliness or a change in the size of the contaminated region.

The above-described guidance information may disappear when a predetermined amount of time passes after the output thereof or when it is determined at the next detection and analysis of cleanliness that the cleanliness has increased above a predetermined level. However, the present disclosure is not limited thereto.

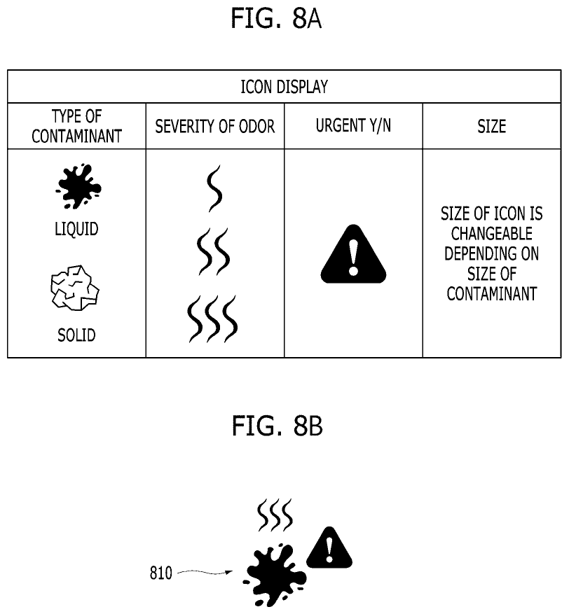

As shown in FIG. 7, in the case in which the guidance information includes an icon-type display object 710, which indicates the occurrence of contamination, the icon may be changed so as to indicate the state of contamination. This will be described below with reference to FIGS. 8A and 8B.

FIGS. 8A and 8B show an example of the contamination guidance information according to an embodiment of the present disclosure.

Referring to FIG. 8A, the shape of a main icon may be determined depending on the type of contaminant, and a first auxiliary icon indicating the severity of odor and a second auxiliary icon indicating the severity of contamination (e.g. the situation in which a contaminated region is expanding or in which the cleanliness is very low) may be displayed near the main icon. In addition, the size of the main icon may be changed depending on the size of the contaminated region. In addition, the color of the main icon may be changed depending on the cleanliness. For example, a blue main icon may indicate high cleanliness, a yellow main icon may indicate intermediate cleanliness, and a red main icon may indicate low cleanliness. However, this is merely illustrative, and the present disclosure is not limited thereto.

Accordingly, in the case in which a region contaminated by liquid having an intermediate contamination level and a strong odor is expanding, the icon 810 may have the shape shown in FIG. 8B.

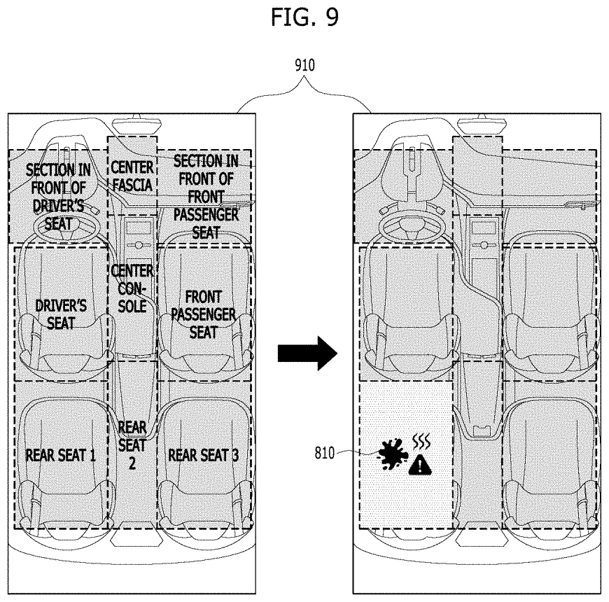

In addition, the guidance information may further include a contaminated region. This will be described below with reference to FIG. 9.

FIG. 9 shows an example of cleanliness-based guidance information for each section according to an embodiment of the present disclosure.

Referring to the left drawing in FIG. 9, the indoor region 910 of the vehicle included in the guidance information may be divided into nine sections, namely, the section in front of the driver's seat (e.g. the steering wheel, the dashboard in front of the driver's seat, the region around the pedal, etc.), the center fascia, the section in front of the front passenger seat (e.g. the glove compartment, the dashboard in front of the front passenger seat, the mat in front of the front passenger seat, etc.), the driver's seat, the center console, the front passenger seat, the rear seat 1, the rear seat 2, and the rear seat 3. As shown in the right drawing in FIG. 9, the controller 140 may display the icon 810, described above with reference to FIGS. 8A and 8B, in the section in which contamination is detected so that the occupant may intuitively recognize which section is contaminated. In addition to the icon 810, a predetermined visual effect (e.g. warning coloration) may be further applied to the section in which contamination is detected.

In addition to detection of indoor contamination and output of guidance information on the detection result, another embodiment of the present disclosure proposes technology for strongly prompting the current user to clean the vehicle and assigning benefits or a penalty to the user depending on the cleaning result.

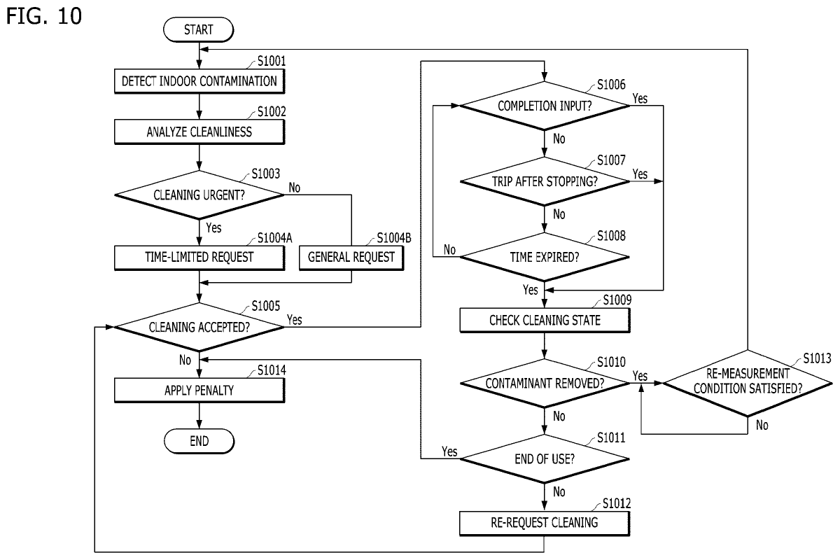

FIG. 10 is a flowchart showing an example of the process of managing the cleanliness of the interior of a vehicle according to another embodiment of the present disclosure.

Referring to FIG. 10, indoor contamination detection (S1001) and cleanliness analysis (S1002) may be performed. Since steps S1001 and S1002 correspond to steps S230 and S240, which have been described above with reference to FIG. 2, a duplicate description thereof will be omitted. The indoor contamination detection process S1001 may be performed in the state in which satisfaction of the starting condition in step S210 and acquisition of the initial environment information in step S220, described above, are assumed. However, the present disclosure is not limited thereto.

Upon determining the occurrence of indoor contamination based on the cleanliness analysis result, the controller 140 may determine whether the need to clean the contaminated region is urgent (S1003). For example, when the cleanliness score, calculated through the method described above with reference to Tables 1 and 2, is less than a preset reference level, the controller 140 may determine that the need to perform cleaning is urgent. Alternatively, the controller 140 may determine whether the need to perform cleaning is urgent based on a cleanliness score calculated based on other criteria. For example, when the cleanliness score is calculated, the controller 140 may perform control such that the cleanliness is weighted more greatly depending on whether the contaminant is liquid, on the material property value, and on the severity of odor than on the size of the contaminated region.

In addition, when determining whether the need to perform cleaning is urgent, the controller 140 may also determine the estimated cleaning time period. The estimated cleaning time period may be determined with reference to Table 3 below.

TABLE 3

| | Factors Required to | |
|---|---|---|
| Contaminant | Calculate Necessary | |
| Category | Time | Calculation Formula |
| Solid | Number of | Number of Contaminants × Time |
| | Contaminants | Necessary to Clean Each |
| | | Contaminant |
| | | + |
| | Size of Particles | Necessary time increases as |
| | | size of particles decreases |
| | | + |
| | Whether or not | Necessary time is weighted |
| | Liquid is Contained | when liquid is contained |
| | | + |
| | Position of | Necessary time is weighted |
| | Contaminant | when contaminant is located |
| | | at corner |
| Liquid | Size of | Size of Contaminated Region × |
| | Contaminated Region | Necessary Cleaning Time for |
| | | Each Size |
| | | + |
| | Number of | Number of Contaminated |
| | Contaminated | Regions × Time Necessary to |
| | Regions | Clean Each Contaminated |
| | | Region |
| | | + |
| | Color Concentration | Necessary time is weighted |
| | | depending on difference |
| | | between color of contaminated |
| | | region and background color |
| | | + |
| | Position of | Necessary time is weighted |
| | Contaminated Region | when contaminated region is |
| | | located at corner |
| Severity of | Odor Concentration | Odor Concentration × Indoor |
| Odor | | Volume of Vehicle |
| | Whether or not | However, when odor-causing |
| | Odor-causing | material has not been |
| | Material Has Been | removed, it is determined |
| | Removed | that cleaning has not been |
| | | performed |

Referring to Table 3, the formula for calculating the estimated cleaning time period may vary depending on the factors required to calculate the necessary time, such as the type of the contaminant (solid or liquid) or the severity of odor. For example, in the case of a solid contaminant, the smaller the size of the particles, the more difficult it is to pick up the same. In this case, therefore, the necessary time is estimated to be long. In the case of a wet solid contaminant (containing liquid), the necessary time is weighted. When the necessary time is estimated based on the severity of odor, the indoor volume of the vehicle is considered. As the indoor volume of the vehicle, a value determined in advance for the vehicle may be used.

The method of estimating the necessary time shown in Table 3 is merely illustrative. It will be apparent to those skilled in the art that various other estimation methods are possible.

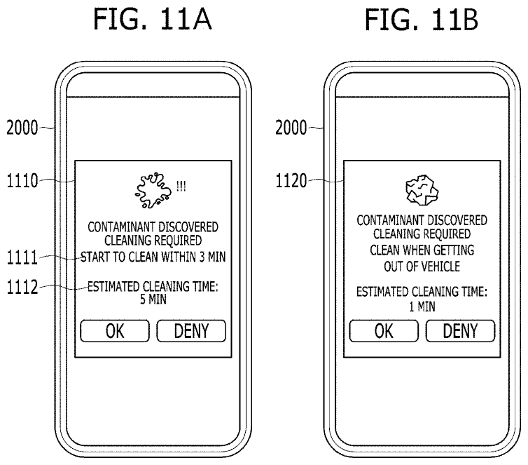

Upon determining that the need to perform cleaning is urgent (Yes in S1003), the controller 140 outputs a time-limited cleaning request so as to designate the time at which to perform cleaning (S1004A). Otherwise (No in S1003), the controller 140 outputs a general cleaning request (S1004B). The time-limited cleaning request or the general cleaning request may be output through the output unit 120 of the vehicle, e.g. the display of the head unit or the cluster, or may be output through a terminal (e.g. a smartphone) carried by the user, which communicates with the communicator 130. Here, the time at which to perform cleaning may be determined depending on the urgency of cleaning. This will be described below with reference to FIGS. 11A and 11B.

FIGS. 11A and 11B show an example of the form in which cleaning request information is output through a terminal of the user according to another embodiment of the present disclosure. In FIGS. 11A and 11B and the drawings below, it is illustrated that various types of display information are output through a terminal 2000 of the user. However, in a similar manner, various types of display information may also be displayed through the output unit 120 of the vehicle.

Referring first to FIG. 11A, when the urgency of cleaning is high, time-limited cleaning request information 1110 may be output through the terminal 2000 of the user. The time-limited cleaning request information 1110 may include an estimated cleaning time 1112 taken to clean the detected contaminant and a time 1111 at which to perform cleaning (hereinafter referred to as a “deadline”).

In contrast, when the urgency of cleaning is low, general cleaning request information 1120 may be output through the terminal 2000 of the user. Unlike the time-limited cleaning request information 1110, the general cleaning request information 1120 may not include a deadline.

In addition, both the time-limited cleaning request information 1110 and the general cleaning request information 1120 may further include the icons indicating contaminant information, which have been described above with reference to FIGS. 8A and 8B.

Referring back to FIG. 10, when the user does not respond to the time-limited cleaning request or the general cleaning request (No in S1005), a penalty may be imposed on the user because the user takes no action to remedy the contamination caused by the user (S1014). The penalty may be at least one of downgrading the membership level in the vehicle-sharing service (a reduction in a discount rate or in a reward rate), an increase in a rental fee for the subject vehicle, subtraction of mileage or accumulated points, or restriction of use of future service, without being limited thereto.

On the other hand, when the user inputs a command indicating acceptance by, for example, pressing an “OK” button included in the time-limited cleaning request information 1110 or the general cleaning request information 1120 (Yes in S1005), the process of determining whether or not cleaning has been performed may be performed.

Specifically, when receiving user input indicating completion of cleaning (Yes in S1006), when the vehicle resumes a trip after stopping (Yes in 1007), or when the time limit in the time-limited cleaning request expires (Yes in S1008) (this step is omitted in the case of the general cleaning request), the controller 140 may check the cleaning state (S1009) and may determine whether the contaminant has been removed based thereon (S1010).

Here, the checking of the cleaning state may mean checking whether the contaminant to be removed has been removed through the above-described indoor contamination detection process. In addition, the removal of the contaminant may mean complete removal of the contaminant, or may mean that the initially detected size, number, or concentration of the contaminants has been reduced by a predetermined rate or more. In addition, in the case of a contaminant having odor, even if the severity of odor has been reduced by cleaning, if an odor-causing material has not been removed, it may be determined that the contaminant has not been removed.

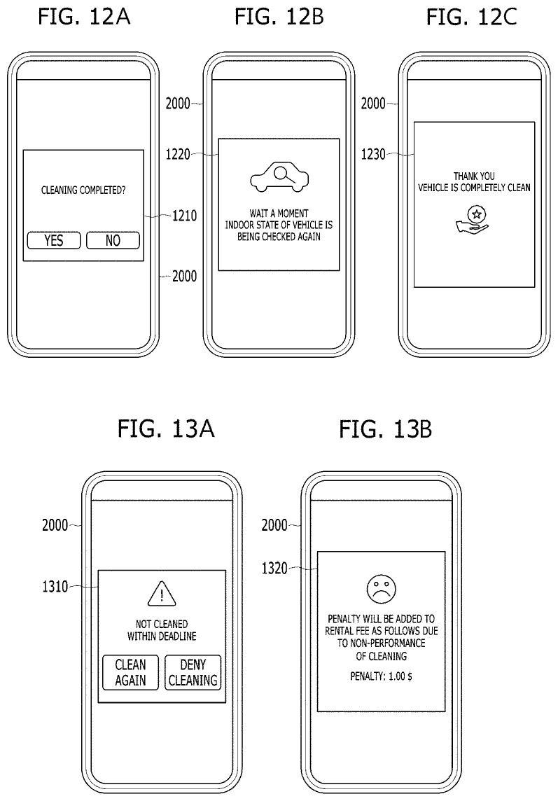

Information output depending on whether cleaning has been performed will be described below with reference to FIGS. 12A, 12B and 12C. FIGS. 12A, 12B and 12C show an example of the form in which information is output through a terminal of the user depending on whether cleaning has been performed according to another embodiment of the present disclosure.

Referring to FIG. 12A, when a predetermined time period (e.g. an estimated cleaning time, a deadline, or the sum thereof) has passed since acceptance of cleaning by the user or when the vehicle resumes a trip after stopping for a predetermined time period or more, a menu 1210 for inputting information on the completion or incompletion of cleaning may be displayed on the terminal 2000 of the user.

When the user selects a button (“Yes”) corresponding to the completion of cleaning, information 1220 indicating that the cleaning state is being checked may be output on the terminal 2000 of the user, as shown in FIG. 12B.

In addition, when it is determined that the contaminant has been removed, information 1230 indicating that cleaning has been completely performed may be displayed, as shown in FIG. 12C. In some embodiments, the information 1230 indicating that cleaning has been completely performed may include information on the benefits (e.g. an increase in mileage or in a discount rate) given to the user due to the completion of cleaning.

Referring back to FIG. 10, when the contaminant has been removed (Yes in S1010), the controller 140 may again determine whether the re-measurement condition is satisfied (S1013). Since step S1013 corresponds to step S270 described above, a duplicate description thereof will be omitted.

On the other hand, when the use of the vehicle ends in the state in which the contaminant is not removed (Yes in S1011), imposition of a penalty is performed (S1014). When the use of the vehicle continues (No in S1011), a cleaning re-request may be performed (S1012). In order to perform imposition of a penalty, the controller 140 may report information on the removal or non-removal of the contaminant to a service server through the communicator 130.

The cleaning re-request may be performed in the manner described above with reference to FIGS. 11A and 11B. However, if the deadline set in the time-limited cleaning request for urgent cleaning has expired, the cleaning re-request may be made in a general cleaning request form.

Information output when the contaminant has not been removed will be described below with reference to FIGS. 13A and 13B. FIGS. 13A and 13B show an example of the form in which information according to non-performance of cleaning is output through the terminal of the user according to another embodiment of the present disclosure.

When the contaminant has not been removed, particularly when the contaminant has not been removed even after the deadline for urgent cleaning has expired, cleaning failure notification information 1310 may be output through the terminal 2000 of the user, as shown in FIG. 13A. When the user has not removed the contaminant until the end of a trip even after the user selected a “Clean Again” button in the notification information 1310 or when the user selects a “Deny Cleaning” button in the notification information 1310, the process of imposing a penalty on the user is performed, and accordingly penalty notification information 1320 may be output, as shown in FIG. 13B.

In another embodiment of the present disclosure described above, the object to be cleaned may correspond only to the contaminant generated after the current user got in the vehicle, or may also correspond to the contaminant left behind by the previous user. When the current user removes only the contaminant generated after the current user got in the vehicle, a penalty may not be imposed on the current user. When the current user also removes the contaminant left behind by the previous user, benefits may be given to the current user. Even if the current user does not remove the contaminant left behind by the previous user, a penalty may not be imposed on the current user.

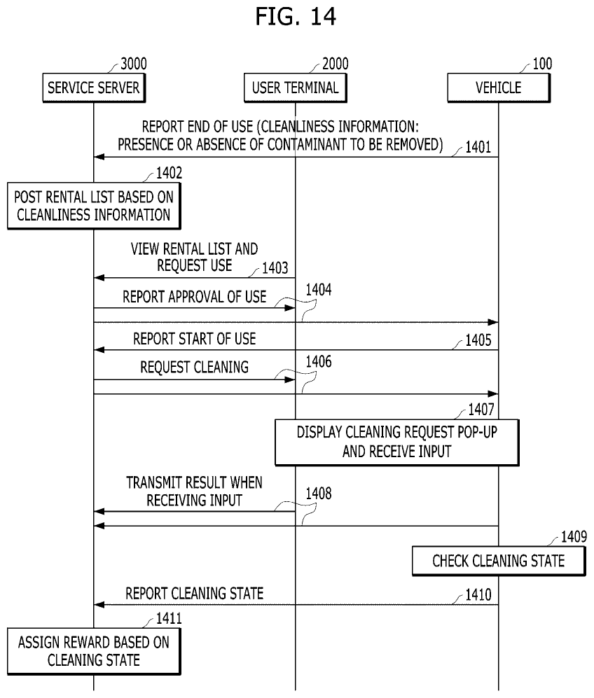

Hereinafter, a process of renting and using a vehicle in a vehicle-sharing service according to another embodiment of the present disclosure will be described with reference to FIGS. 14, 15A, 15B, 15C, 15D, and 16. For convenience of explanation, the following description will be made on the assumption that the previous user has not removed a contaminant at the end of use of a vehicle and thus the contaminant remains in the interior of the vehicle.

FIG. 14 is a flowchart showing an example of the process in which the next user uses a shared vehicle in the state in which a contaminant left behind by the previous user is present in the interior of the shared vehicle according to another embodiment of the present disclosure.

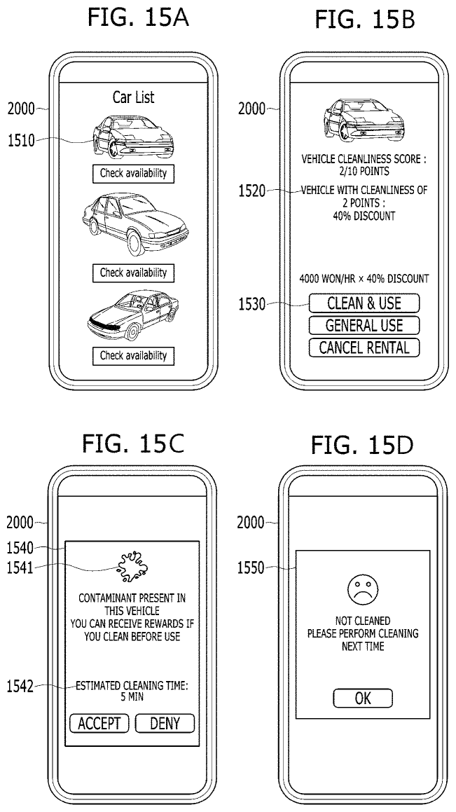

FIGS. 15A, 15B, 15C and 15D show an example of information output through the terminal of the user in the process of using a shared vehicle in which a contaminant remains according to another embodiment of the present disclosure.

Referring to FIGS. 14, 15A, 15B, 15C, and 15D, when the use of the vehicle by the previous user ends, the vehicle 100 reports the end of use thereof to a vehicle-sharing service server 3000 (hereinafter referred to as a “service server”) through the communicator 130 (S1401). When the end of use of the vehicle 100 is reported, information on the final cleanliness (the presence or absence of a contaminant to be removed), which was measured after the end of the previous trip of the vehicle 100, may also be transmitted to the service server.

Considering the cleanliness information, the service server 3000 may register and post a list of available vehicles (i.e. a rental list) in a form such that the user is capable of checking the corresponding vehicle 100 through the terminal 2000 of the user (S1402). Here, the consideration of the cleanliness information may mean determination of benefits given to the next user who will use the corresponding vehicle in spite of the remaining contaminant.

When the corresponding vehicle is posted on the rental list, the user may access the service server through the terminal 2000 of the user to view the rental list, and may request use of the corresponding vehicle 100 (S1403). For example, the rental list shown in FIG. 15A may be displayed on the terminal 2000 of the user. In this case, when an item 1510 corresponding to the vehicle 100 is selected, the cleanliness level of the vehicle 100 and benefit information 1520 may be displayed, as shown in FIG. 15B. The user may express his/her intention to clean the vehicle by selecting a “Clean & Use” menu 1530. When the user does not intend to clean the vehicle, the user may select a “General Use” menu.

In response to the request for use by the user, the service server 3000 may check a fee-charging method and may approve the use of the vehicle 100. Thereafter, the service server 3000 may transmit the result of use approval to at least one of the terminal 2000 of the user or the vehicle 100 (S1404).

Subsequently, when the next user starts to use the vehicle 100, for example, when the door of the vehicle is opened or the vehicle is started, the vehicle 100 may transmit a use start report to the service server 3000 (S1405).

Upon receiving the use start report, the service server 3000 may transmit a cleaning request to at least one of the terminal 2000 of the user or the vehicle 100 in the case in which the user expressed his/her intention to clean the vehicle 100 when requesting use of the same (S1406). In some embodiments, the cleaning request may also be transmitted in the case in which the user selected general use when requesting use of the vehicle 100.

Upon receiving the cleaning request, at least one of the terminal 2000 of the user or the vehicle 100 may display a cleaning request pop-up in order to induce the user to perform cleaning, and may wait for input (S1407). For example, as shown in FIG. 15C, when the cleaning request pop-up 1540 is output through the terminal 2000 of the user, the cleaning request pop-up 1540 may include an icon 1541 indicating the state of a contaminant, an estimated cleaning time 1542, and information on benefits (rewards) to be given to the user.

When the user inputs “Accept” or “Deny” in the cleaning request pop-up, the terminal 2000 of the user or vehicle 100 may receive the user input and may transmit information on the received input to the service server 3000 (S1408).

Subsequently, the vehicle 100 may check the cleaning state (S1409), and may transmit information on the cleaning state to the service server 3000 (S1410). The service server 3000 may perform reward processing on the user based on the information on the cleaning state (S1411). The reward level may be determined differently depending on the contamination level of the removed contaminant, the time taken to clean the vehicle, and the cleaned area. However, even though the user accepted the cleaning request, if the user has not removed a contaminant, the user may receive only the reward, such as a discount, notified in advance when requesting use of the vehicle 100.

The process of checking the cleaning state (S1409) may be performed before the use of the vehicle by the user ends, or may be performed after the use of the vehicle by the user ends. Alternatively, when the user inputs the completion of cleaning through the menu 1210 shown in FIG. 12A, the process of checking the cleaning state (S1409) may be performed.

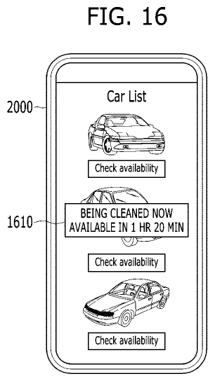

With regard to a vehicle that has not been continuously selected by users due to the low cleanliness score thereof, a vehicle that has not been continuously cleaned by users who used the vehicle in turn, or a vehicle contaminated below a preset cleanliness level, the service server 3000 may search for a cleaning company and may transmit a cleaning request to the cleaning company together with the location of the vehicle and the cleanliness information. In this case, the rental list may be managed as shown in FIG. 16.

FIG. 16 shows another example of the form in which information is output through the terminal of the user in the process of using a shared vehicle in which a contaminant remains according to another embodiment of the present disclosure.

Referring to FIG. 16, with regard to a vehicle contaminated below a preset cleanliness level, information 1610 indicating that the corresponding vehicle is being cleaned may be included in the rental list displayed on the terminal 2000 of the user. An estimated waiting time may be displayed in the information 1610. The estimated waiting time may be a time taken for the vehicle to be capable of being reused, which may be calculated based on a time taken to clean the vehicle and a vehicle moving time (a time taken to search for a cleaning company and a time taken for the vehicle to move to and return from the cleaning company).

The present disclosure may be implemented as code that can be written on a computer-readable recording medium and thus read by a computer system. The computer-readable recording medium includes all kinds of recording devices in which data that may be read by a computer system are stored. Examples of the computer-readable recording medium include a Hard Disk Drive (HDD), a Solid-State Disk (SSD), a Silicon Disk Drive (SDD), a Read-Only Memory (ROM), a Random Access Memory (RAM), a Compact Disk ROM (CD-ROM), a magnetic tape, a floppy disc, and an optical data storage.

As is apparent from the above description, a vehicle according to at least one embodiment of the present disclosure configured as described above is capable of determining an indoor contaminated region in various manners and analyzing the contamination level.

In addition, it is possible to induce a user to clean the vehicle by applying information on detected indoor contamination to the reputation of the user and to a rental fee.

However, the effects achievable through the disclosure are not limited to the above-mentioned effects, and other effects not mentioned herein will be clearly understood by those skilled in the art from the above description.

It will be apparent to those skilled in the art that various changes in form and details may be made without departing from the spirit and essential characteristics of the disclosure set forth herein. Accordingly, the above detailed description is not intended to be construed to limit the disclosure in all aspects and to be considered by way of example. The scope of the disclosure should be determined by reasonable interpretation of the appended claims and all equivalent modifications made without departing from the disclosure should be included in the following claims.

What is claimed is:

1. A method of managing cleanliness of an interior of a vehicle, the method comprising:
detecting indoor contamination using a contamination detector comprising at least a camera;
outputting information on a cleaning request upon detecting a first contaminant as a result of the detecting the indoor contamination;
determining whether the first contaminant has been removed when a predetermined condition is satisfied; and
imposing a penalty on a user upon determining that the first contaminant has not been removed as a result of the determining.

2. The method according to claim 1, further comprising determining a degree of urgency of cleaning of the first contaminant,
wherein the outputting comprises outputting, based on the determined degree of urgency, a first cleaning request or a second cleaning request comprising a deadline determined based on the determined degree of urgency.

3. The method according to claim 2, wherein the first contaminant comprises a contaminant generated after the user gets in the vehicle.

4. The method according to claim 2, wherein the predetermined condition comprises at least one of a case in which a cleaning completion command is input, a case in which the vehicle resumes a trip after stopping, or a case in which the deadline has expired.

5. The method according to claim 1, wherein the imposing a penalty is performed when an input indicating refusal to comply with the cleaning request is received or when the first contaminant is not removed before an end of use of the vehicle after the user accepts the cleaning request.

6. The method according to claim 1, further comprising determining an estimated cleaning time for the first contaminant,
wherein the cleaning request comprises the estimated cleaning time.

7. The method according to claim 1, further comprising:
receiving a cleaning request for a second contaminant generated by a previous user from an outside; and
outputting the received cleaning request for the second contaminant.

8. The method according to claim 7, further comprising:
determining whether the second contaminant has been removed in response to the cleaning request for the second contaminant; and
giving a reward to the user when the second contaminant has been removed.

9. The method according to claim 1, wherein the detecting indoor contamination comprises:
detecting at least one object in an indoor region subjected to contamination detection; and
detecting whether contamination has occurred in the indoor region excluding a region in which the at least one object is detected.

10. A none-transitory computer-readable recoding medium containing a program configured to perform a method of managing a cleanliness of an interior of a vehicle, wherein the method comprises:
detecting indoor contamination using a contamination detector comprising at least a camera;
outputting information on a cleaning request upon detecting a first contaminant as a result of the detecting the indoor contamination;
determining whether the first contaminant has been removed when a predetermined condition is satisfied; and
imposing a penalty on a user upon determining that the first contaminant has not been removed as a result of the determining.

11. A device for managing cleanliness of an interior of a vehicle, the device comprising:
an output unit;
a contamination detector comprising at least a camera; and
a controller configured to:
determine indoor contamination based on information acquired using the contamination detector,
output information on a cleaning request through the output unit when a first contaminant is present,
determine whether the first contaminant has been removed using the contamination detector when a predetermined condition is satisfied, and
perform control such that a penalty is imposed on a user upon determining that the first contaminant has not been removed.

12. The device according to claim 11, wherein the controller determines a degree of urgency of cleaning of the first contaminant and performs control such that a first cleaning request or a second cleaning request comprising a deadline determined based on the determined degree of urgency is output based on the determined degree of urgency.

13. The device according to claim 12, wherein the first contaminant comprises a contaminant generated after the user gets in the vehicle.

14. The device according to claim 12, wherein the predetermined condition comprises at least one of a case in which a cleaning completion command is input, a case in which the vehicle resumes a trip after stopping, or a case in which the deadline has expired.

15. The device according to claim 11, wherein the controller performs control such that the penalty is imposed on the user when an input indicating refusal to comply with the cleaning request is received or when the first contaminant is not removed before an end of use of the vehicle after the user accepts the cleaning request.

16. The device according to claim 11, wherein the controller determines an estimated cleaning time for the first contaminant, and
wherein the cleaning request comprises the estimated cleaning time.

17. The device according to claim 11, further comprising a communicator configured to receive a cleaning request for a second contaminant generated by a previous user from an outside,
wherein the controller performs control such that the received cleaning request for the second contaminant is output through the output unit.

18. The device according to claim 17, wherein the controller determines whether the second contaminant has been removed in response to the cleaning request for the second contaminant using the contamination detector, and performs control such that a reward is given to the user when the second contaminant has been removed.

19. The device according to claim 11, wherein the controller detects at least one object in an indoor region subjected to contamination detection and detects whether contamination has occurred in the indoor region excluding a region in which the at least one object is detected.
